(19) United States
(12) Patent Application Publication
(10) Pub. No.: US 2023/0414094 A1
(43) Pub. Date: Dec. 28, 2023

(54) VOLUME-BASED LAYER-INDEPENDENT FRAMEWORK FOR DETECTION OF RETINAL PATHOLOGY

(71) Applicant: Oregon Health & Science University, Portland, OR (US)

(72) Inventors: Shaohua Pi, Portland, OR (US); Yali Jia, Portland, OR (US)

(73) Assignee: Oregon Health & Science University, Portland, OR (US)

(21) Appl. No.: 18/211,116

(22) Filed: Jun. 16, 2023

Related U.S. Application Data
(60) Provisional application No. 63/354,990, filed on Jun. 23, 2022.

(51) Int. Cl. A61B 3/10; G06T 7/11; G06T 7/00

(52) U.S. Cl. CPC A61B 3/102; G06T 7/11; G06T 7/0016; G06T 2207/10101; G06T 2211/404

(57) ABSTRACT

Disclosed herein is a method for detecting retinal pathologies in three dimensions using structural and angiographic OCT. The method in accordance with the present disclosure may operate by detecting deviations in reflectance and perfusion from a depth-normalized standard retina created by merging and averaging scans from healthy subjects. In one example, the deviations from the standard retina highlight key pathologic features, while depth-normalization obviates the need to segment retinal layers. Additionally, a composite pathology index is disclosed herein that measures average deviation from the standard retina. The present method is amenable to automation and may be implemented in an integrated system and/or provided in the form of software encoded on a computer-readable medium.

### CROSS-REFERENCE TO RELATED APPLICATION

The present application claims priority to U.S. Provisional Patent Application No. 63/354,990, filed Jun. 23, 2022, entitled “A VOLUME BASED LAYER-INDEPENDENT FRAMEWORK FOR DETECTION OF RETINAL PATHOLOGY”, the entire disclosure of which is hereby incorporated by reference.

### ACKNOWLEDGEMENT OF GOVERNMENT SUPPORT

This invention was made with government support under R01 EY027833 and R01 EY024544 awarded by the National Institutes of Health. The government has certain rights in the invention.

### TECHNICAL FIELD

The present disclosure generally relates to the field of ophthalmology. In particular, apparatuses, systems, and methods for the detection and characterization of retinal pathologies are disclosed.

### BACKGROUND

Pathology can occur anywhere throughout the complex, layered structure of the retina. Traditional ophthalmic imaging modalities such as color fundus photography and fluorescein angiography are two-dimensional and hence cannot depth-resolve features, meaning that they may fail to elucidate important relationships between the location of pathology and vision. In contrast, optical coherence tomography (OCT) and its angiography (OCTA) are 3-dimensional (3D) imaging modalities capable of supplying a volumetric description of the retina. Nonetheless, in practice most OCT and OCTA data is graded and analyzed using two-dimensional (2D) data representations such as cross-sectional or en face images. Since a single OCT volume typically contains hundreds of cross sections (each of which must be processed with retinal layer segmentation to produce en face images), contemporary OCT analysis can be prohibitively time consuming. Furthermore, 2D reductions of the full OCT volume may obscure relationships between disease progression and the (volumetric) location of pathology within the retina and can be prone to mis-segmentation artifacts. Therefore, there still exists a need for reliable systems and methods that can detect retinal pathologies in three dimensions.

### BRIEF DESCRIPTION OF THE SEVERAL VIEWS OF THE DRAWINGS

The patent or application file contains at least one drawing/photograph executed in color. Copies of this patent or patent application publication with color drawings(s)/photograph(s) will be provided by the Office upon request and payment of the necessary fee.

Embodiments will be readily understood by the following detailed description in conjunction with the accompanying drawings and the appended claims. Embodiments are illustrated by way of example and not by way of limitation in the figures of the accompanying drawings.

FIG. 1 depicts a standardized retinal volume registered and averaged from healthy subjects with representative cross-sectional structural (upper-right corner) and angiographic (lower-right corner) B-scans. ILM refers to inner limiting membrane, NFL refers to nerve fiber layer, GCL refers to ganglion cell layer, IPL refers to inner plexiform layer, INL refers to inner nuclear layer, OPL refers to outer plexiform layer, ONL refers to outer nuclear layer, EZ refers to ellipsoid zone, and BM refers to Bruch's membrane. NFLP refers to nerve fiber layer plexus, SVP refers to superficial vascular plexus, SVC refers to superficial vascular complex, ICP refers to intermediate capillary plexus, DCP refers to deep capillary plexus, and CC refers to choroid capillary.

FIGS. 2A-2B depict percent retinal depth for retinal layer boundaries in the macular region (6×6-mm centered at foveal pit), in accordance with various embodiments. FIG. 2A illustrates color coding by retinal layer boundaries, and FIG. 2B illustrates color coding by percent retinal depth in heathy subjects. ILM refers to inner limiting membrane, NFL refers to nerve fiber layer, GCL refers to ganglion cell layer, IPL refers to inner plexiform layer, INL refers to inner nuclear layer, OPL refers to outer plexiform layer, ONL refers to outer nuclear layer, EZ refers to ellipsoid zone, and BM refers to Bruch's membrane.

FIG. 3A depicts population variation displayed as a retinal thickness standard deviation map generated from healthy subjects. FIG. 3B illustrates comparison of a merged retina with and without axial normalization, with axial profiles shown at the location of the white dashed line in the B-scan. The different retinal layers are more readily identifiable with normalization, and layer boundaries are sharper. FIG. 3C illustrates representative retinal vascularization along depth at the location of white dashed line in the B-scan in FIG. 3B. FIG. 3D depicts voxel-wise vessel density map demonstrated on a B-scan and an axial profile at the location of the white dashed line in the B-scan.

FIGS. 4A-4D illustrate detection of hyper-reflective paracentral acute middle maculopathy (PAMM) in an eye with retinal artery occlusion (RAO), in accordance with an embodiment of the present disclosure. FIG. 4A illustrates a structural B-scan.

FIG. 4B illustrates a structural en face image projected from the INL slab. The white dashed line in FIG. 4B indicates the position of B-scan in FIG. 4A. FIG. 4C shows a B-scan and FIG. 4D illustrates en face image of hyper-reflective pathology color-coded by percent retinal depth (ILM:O, BM:100).

FIG. 5 illustrates detection of hyper-reflective retinal pathologies including extended paracentral acute middle maculopathy (PAMM) in retinal artery occlusion (RAO), hard exudates in diabetic retinopathy (DR), and drusen in age-related macular degeneration (AMD), respectively. Colors are coded by percent retinal depth in the B-scans, and projected by center of mass for en face display.

FIGS. 6A-6F illustrate detection of hypo-reflective retinal pathology in diabetic retinopathy (DR), in accordance with an embodiment of the present disclosure. FIG. 6A illustrates a structural B-scan showing intraretinal fluid and subretinal fluid.

FIG. 6B illustrates a structural en face image, wherein the white dashed line indicates the position of B-scan in FIG. 6A. FIG. 6C shows a B-scan and FIG. 6D illustrates en face image of hypo-reflective pathology color-coded by percent retinal depth (ILM:O, BM:100). FIG. 6E shows an enlarged version of the en face image of FIG. 6D indicating intraretinal fluid and subretinal fluid. FIG. 6F illustrates hypo-reflectance volume along a grading score to indicate severity of diabetic retinopathy (DR).

FIGS. 7A-7L illustrate detection of retinal non-perfusion in three dimensions. FIGS. 7A, 7B, 7C, 7D, and 7E are representative B-scans of the angiogram volume (7A: merged reference retina; 7C: diseased retina) and the perfusion volume (7B: merged reference retina; 7D: diseased retina) simulated by the convolution of the angiogram volume with a 3-D Gaussian kernel, as well as the detected non-perfusion (7E) calculated through a comparison between the reference retina and the diseased retina, and color coded by percent retinal depth. FIGS. 7F, 7G, and 7H are corresponding B-scan of the structure (7F), the overlay of non-perfusion with the structure (7G), and the overlay of non-perfusion with the angiogram (7H), respectively. FIGS. 7I, 7J, and 7K illustrate the en face image of retinal vasculature showing the capillary dropout (7I), together with the en face images of the detected non-perfusion areas (7J) and overlay (7K). FIG. 7L illustrates volumetric visualization of the detected non-perfusion volume. Location of the representative B-scans is indicated with the dashed line in FIG. 7I.

FIG. 8 depicts visualization of choroidal neovascularization (CNV) in an eye with age-related macular degeneration (AMD) detected with a pathologic angiogenesis enhancement (PAE) algorithm. For example, FIG. 8 illustrates a volumetric visualization of the raw angiogram, a volumetric visualization of a pathological angiogenesis enhanced (PAE) angiogram revealing CNV, and an en face visualization of the PAE angiogram delineating the CNV.

FIGS. 9A-9C depict en face visualization of choroidal neovascularization (CNV), in accordance with various embodiments. FIG. 9A illustrates CNV in raw angiograms. FIG. 9B illustrates CNV in projection resolved (PR) angiograms. FIG. 9C illustrates CNV in the proposed pathological angiogenesis enhanced (PAE) angiograms.

FIGS. 10A-10B illustrate diagnostic power of various pathology indexes for diabetic retinopathy (DR). As depicted in FIG. 10A, the combined pathology index achieved a R=0.95 Spearman correlation coefficient with the DR severity. The black dotted line indicates a pathology index cutoff that correctly diagnoses referable DR in the majority of cases. FIG. 10B illustrates receiver operating characteristic curves for the pathology index and specific components for referable vs non-referable DR classification.

FIGS. 11A-11D illustrate scans and plots, in accordance with Example 1 described herein. FIG. 11A depicts automatically detected and manually labeled foveal centers in a representative scan. FIG. 11B illustrates that the automated detection method for the foveal center may be effective, with a median distance of 59 μm from the manually labeled center in n=1519 scans. FIG. 11C indicates that lateral registration may be achieved by foveal center alignment in a 9×9-mm image, with the nasal side flipped in left eyes. FIG. 11D illustrates that the axial normalization may be achieved by calculating the percent retinal depth.

FIG. 12A illustrates en face binary images which demonstrate vascular, avascular and CNV A-lines in a scan. FIG. 12B illustrates the distributions of angiogram decorrelation values for the three types of A-lines over the entire scan and at specific locations, in accordance with Example 1 described herein.

FIG. 13 schematically shows an example system for processing OCT datasets in accordance with the disclosure.

FIG. 14 schematically shows an example of a computing system in accordance with the disclosure.

FIG. 15 illustrates a flow chart of an example method for detecting retinal pathologies, in accordance with various embodiments of the present disclosure.

### DETAILED DESCRIPTION OF DISCLOSED EMBODIMENTS

Disclosed herein is a three-dimensional analytic framework to detect a full spectrum of retinal pathologies volumetrically using structural and angiographic OCT. The methods according to the present disclosure operates by detecting deviations in a target retina from a depth-normalized reference volume (also referred to as a standard reti created by averaging scans from healthy subjects. The deviations may highlight key pathologic features including, but not limited to, abnormal reflectivity, neovascularization, and non-perfusion. In embodiments, the present method requires minimal retinal layer segmentations. The present disclosure also provides a composite pathology index that measures average deviation from the standard retina in various categories. In embodiments, the pathology index correlates with diabetic retinopathy (DR) severity. Thus, the present method has potential both as a visual interpretive aid as well as a foundation for quantitative analysis.

An example method for detecting retinal pathology using the disclosed subject matter may include: constructing a standard retina volume by averaging a plurality of depth-normalized scans from healthy subjects; registering a target scan volumetrically from a diseased retina; comparing voxel-wise the target scan to the standard retina volume to detect one or more deviations; and detecting one or more retinal pathologies in the target scan based on the one or more deviations detected during comparison of the target scan with the standard retina volume.

The embodiments described herein may provide an automated system for diagnosing conditions in the retina and for differentiating pathologic and non-pathologic conditions. The disclosed methods and systems may be integrated into commercial OCT systems to potentially improve ophthalmic research and clinical care. Further embodiments also include a computer-readable medium encoding executable instructions to perform the disclosed methods.

The systems and methods of the present disclosure establish a reference standard retina to aid in identification of pathologic deviations due to disease. The present systems and methods enable volumetric comparison and merging of OCT scans from different subjects and enable detection of retinal pathologies voxel-wise. Furthermore, the disclosed systems and methods combine a full spectrum of clinical features for fast screening.

In the following detailed description, reference is made to the accompanying drawings which form a part hereof, and in which are shown by way of illustration embodiments that can be practiced. It is to be understood that other embodiments can be utilized and structural or logical changes can be made without departing from the scope. Therefore, the following detailed description is not to be taken in a limiting sense, and the scope of embodiments is defined by the appended claims and their equivalents.

Various operations can be described as multiple discrete operations in turn, in a manner that can be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

In various embodiments, structure and/or flow information of a sample can be obtained using OCT (structure) and OCT angiography (flow) imaging based on the detection of spectral interference. Such imaging can be two-dimensional (2-D) or three-dimensional (3-D), depending on the application. Structural imaging can be of an extended depth range relative to prior methods, and flow imaging can be performed in real time. One or both of structural imaging and flow imaging as disclosed herein can be enlisted for producing 2-D or 3-D images.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

In order to facilitate review of the various embodiments of the disclosure, the following explanation of specific terms is provided:

A-scan: A reflectivity profile that contains information about spatial dimensions and location of structures with an item of interest (e.g., an axial depth scan). An A-scan is an axial scan directed along the optical axis of the OCT device and penetrates the sample being imaged. The A-scan encodes reflectivity information (for example, signal intensity) as a function of the depth of the sample being imaged. The A-scan encodes reflectivity information (for example, signal intensity) as a function of depth.

B-scan: A cross-sectional tomograph that can be achieved by laterally combining a series of axial depth scans (e.g., A-scans). A B-scan encodes planar cross-sectional information from the sample and is typically presented as an image. Thus, a B-scan can be called a cross sectional image.

Dataset: As used herein, a dataset is an ordered-array representation of stored data values that encodes relative spatial location in row-column-depth (x-y-z axes) format. In the context of OCT, as used herein, a dataset can be conceptualized as a three dimensional array of voxels, each voxel having an associated value (for example, an intensity value or a decorrelation value). An A-scan corresponds to a set of collinear voxels along the depth (z-axis) direction of the dataset; a B-scan is made up of set of adjacent A-scans combined in the row or column (x- or y-axis) directions. Such a B-scan can also be referred to as an image, and its constituent voxels referred to as pixels. A collection of adjacent B-scans can be combined to form a 3D volumetric set of voxel data referred to as a 3D image. In the system and methods described herein, the dataset obtained by an OCT scanning device is termed a “structural OCT” dataset whose values can, for example, be complex numbers carrying intensity and phase information. This structural OCT dataset can be used to calculate a corresponding dataset termed an “OCT angiography” dataset of decorrelation values reflecting flow within the imaged sample. There is a direct correspondence between the voxels of the structural OCT dataset and the OCT angiography dataset. Thus, values from the datasets can be “overlaid” to present composite images of structure and flow (e.g., tissue microstructure and blood flow) or otherwise combined or compared.

En Face angiogram/image: OCT angiography data can be presented as a projection of the three dimensional dataset onto a single planar image called an en face angiogram (Wallis J et al, Med Imaging IEEE Trans 8, 297-230 (1989); Wang R K et al, 2007 supra; Jia Y et al, 2012 supra; incorporated by reference herein). Construction of such an en face angiogram requires the specification of the upper and lower depth extents that enclose the region of interest within the retina OCT scan to be projected onto the angiogram image. These upper and lower depth extents can be specified as the boundaries between different layers of the retina (e.g., the voxels between the inner limiting membrane and outer plexiform layer can be used to generate a 2D en face angiogram of the inner retina). Once generated, the en face angiogram image can be used to quantify various features of the retinal vasculature as described herein. This quantification typically involves the setting of a threshold value to differentiate, for example, the pixels that represent active vasculature from static tissue within the angiogram. These 2D en face angiograms can be interpreted in a manner similar to traditional angiography techniques such as fluorescein angiography (FA) or indocyanine green (ICG) angiography, and are thus well-suited for clinical use. It is also common to generate en face images from structural OCT data in a manner analogous to that used to generate en face angiograms. Angiograms from different layers may also be color-coded and overlaid to present composite angiograms with encoded depth information; structural en face images may also be included in such composite image generation.

Optical coherence tomography (OCT) is an optical signal acquisition and processing method which is capable of capturing micrometer-resolution, three-dimensional images from within optical scattering media, e.g., biological tissue. Optical coherence tomography is based on interferometric techniques and typically employs near-infrared light. The use of relatively long wavelength light allows it to penetrate into the scattering medium. As remarked above, among its many applications, OCT-based ocular imaging has found widespread clinical use and can be performed quickly and easily with minimal expertise. OCT is a non-invasive imaging modality which provides accurate and precise anatomical reproduction of the retinal layers thus is well suited for use in detecting and diagnosing diseases of the retina.

The human retina is a light-sensitive tissue located between the vitreous and the choroid. The most prevalent clinical imaging modality for evaluating the retina is optical coherence tomography (OCT) due to its three-dimensional (3D), non-invasive nature and micron-scale resolution (Huang, D. et al. Optical coherence tomography. Science 254, 1178-1181 (1991), incorporated by reference herein). As a functional extension of OCT, OCT angiography (OCTA) is an emerging technology that, when combined with structural OCT, enables simultaneous visualization of retinal structure and vascular morphology without requiring intravenous dye for contrast (e.g., as described in Jia, Y et al. Quantitative optical coherence tomography angiography of vascular abnormalities in the living human eye. Proceedings of the National Academy of Sciences 112, E2395-E2402 (2015), incorporated by reference herein). Using structural OCT and OCTA, highly-organized laminar layers spanning the entire retina from the inner limiting membrane (ILM) to Bruch's membrane (BM), the avascular foveal pit in the macula, and vascular plexuses supplying specific anatomic slabs can all be observed simultaneously in a data volume obtained during a single procedure. Structural OCT and OCTA measurements (e.g., layer thicknesses, neovascularization, non-perfusion areas, or vessel densities) can be extracted through post-processing and used for disease diagnosis (e.g., as described in Spaide, R F et al. Optical coherence tomography angiography. Progress in retinal and eye research 64, 1-55 (2018), incorporated by reference herein).

Although structural OCT and OCTA provide volumetric data, both human graders and algorithms usually analyze cross-sections or en face images constructed by projecting the OCT/OCTA signals across a retinal slab. Analytic approaches that rely on 2D reductions of the full 3D data volume will often incur some disadvantages; for OCT these include requisite anatomic layer segmentation when en face images are needed and the potential for 2D representations to distort the important qualities of some features that are best captured in 3D. On the other hand, a full 3D characterization of structural (e.g. retinal fluid volume) and vascular pathologies (e.g. 3D parafoveal vessel density) is more sensitive for classifying and staging diseases than the same biomarkers characterized only in 2D. Nonetheless, volumetric characterization of pathology remains under-utilized, which is in part attributable to the difficulty of visualizing and analyzing 3D data.

To address this issue, an improved framework has been developed and disclosed herein that can readily detect retinal pathology and visualize its location in 3D. This framework relies on comparing target scans to a standard retina volume constructed by averaging scans from several healthy retinas. Volumetric registration, merger, and comparison of macular scans from different subjects may be achieved by normalizing to percent depth within the retina and laterally aligning foveal avascular zones (FAZ). This process requires segmentation of just two anatomic layer boundaries, which can be compared to the many OCT algorithms that require segmentation of several anatomic layers. The method disclosed herein also smears out variation between individual healthy retinas such as differences in retinal thickness and curvature. For this reason, target OCT scans can then be compared to the standard retina in order to detect disparities, with large deviations corresponding to likely pathologic features. A wide spectrum of clinical features including, but not limited to, abnormal reflectivity, neovascularization, and non-perfusion may be detected and visualized according to the methods of the present disclosure. In addition, a novel pathology index has been constructed and disclosed herein that measures the average deviation of a target retina from the standard. This pathology index has been shown herein to correlate with diabetic retinopathy (DR) severity. Therefore, the disclosed framework has potential as both a visual/interpretive aid and as a foundation for quantitative analysis.

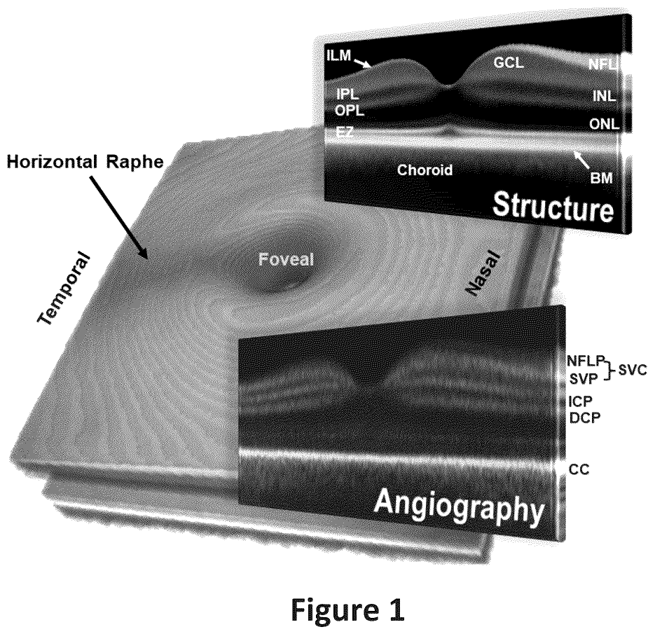

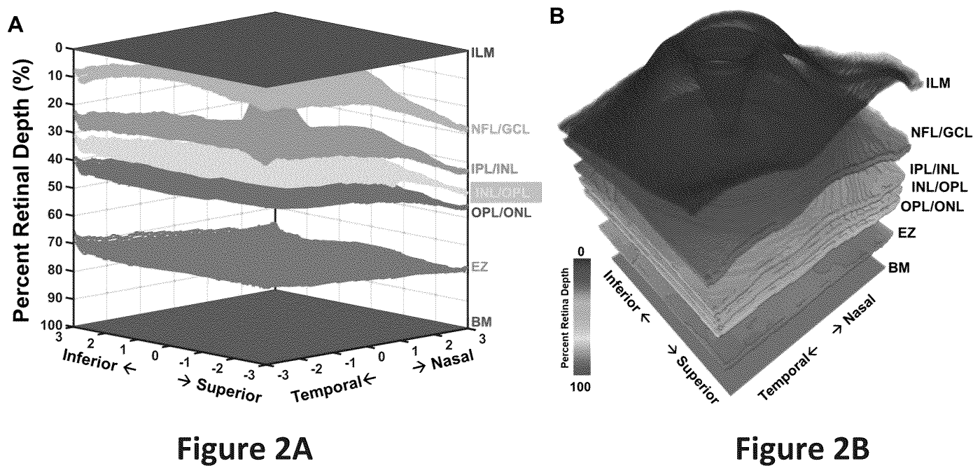

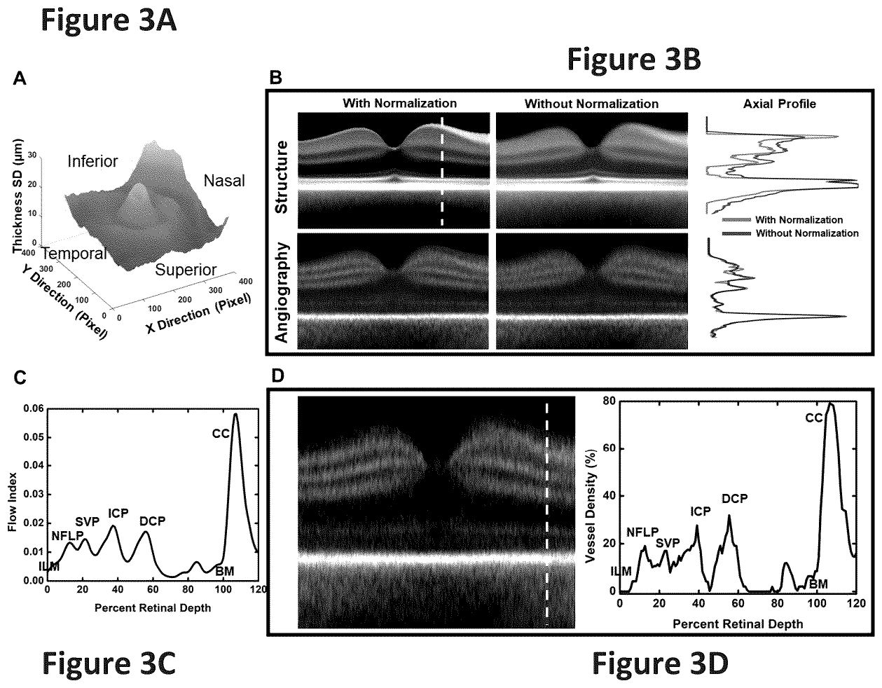

FIG. 1 illustrates a standardized retinal volume registered and averaged from healthy subjects with representative cross-sectional structural (upper-right corner) and angiographic (lower-right corner) B-scans. To obtain the standard retina used in this work, structural OCT and OCTA volumes from healthy subjects may be registered volumetrically by laterally aligning the foveal center and normalizing each A-line to percent retinal depth (ILM: 0, BM: 100). Transforming to depth normalized coordinates, as shown in FIGS. 2A-2B, help to eliminate inherent variation in retinal thickness and curvature that would otherwise lead to blurry images carrying imprecise anatomic information. Registered in this way, as illustrated in FIG. 3A, contrast between retinal layers in the structural OCT channel was enhanced instead (FIG. 3B). In the standard retina (e.g., shown in FIG. 1) the horizontal raphe is clearly visible, indicating morphology may be dramatically enhanced by registering the normal controls in a standard volume.

Both the merged structural and angiographic standard retinas may be interpreted as probabilities maps, with the structural volume yielding the probability that a region is highly reflective and the angiographic standard giving the probability that a region is vascularized. However, while the structural standard retina retains the appearance of a scan from a single individual, the angiographic standard retina does not since blood vessel morphology is unique for each individual. Instead, the superficial vascular complex (SVC), intermediate capillary plexus (ICP), and deep capillary plexus (DCP) look like bands, rather than a collection of vessels (e.g., as shown in FIG. 1 and FIGS. 3A-3D). The angiographic standard retina volume does, however, correspond closely to a voxel-wise 3D vessel density map (FIG. 3D).

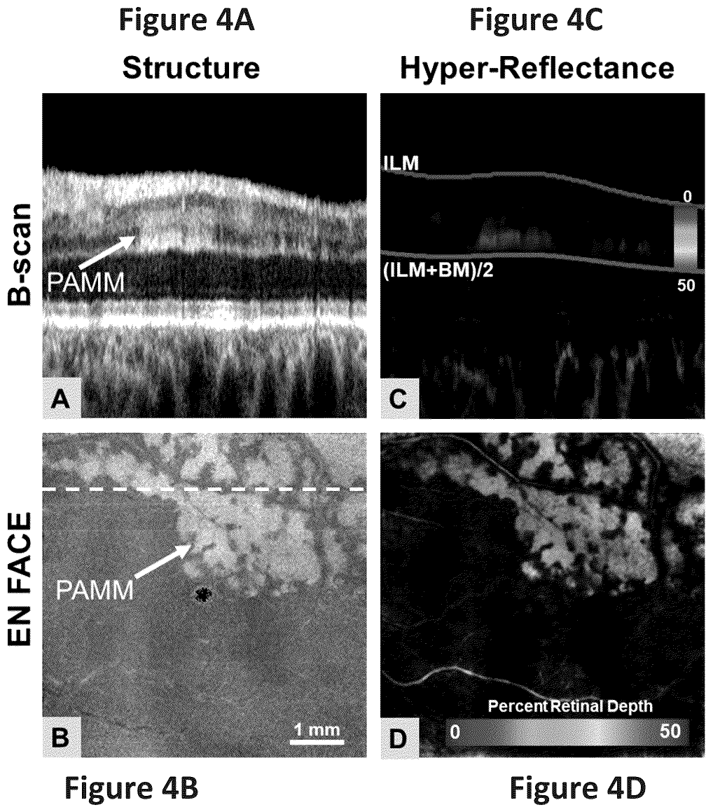

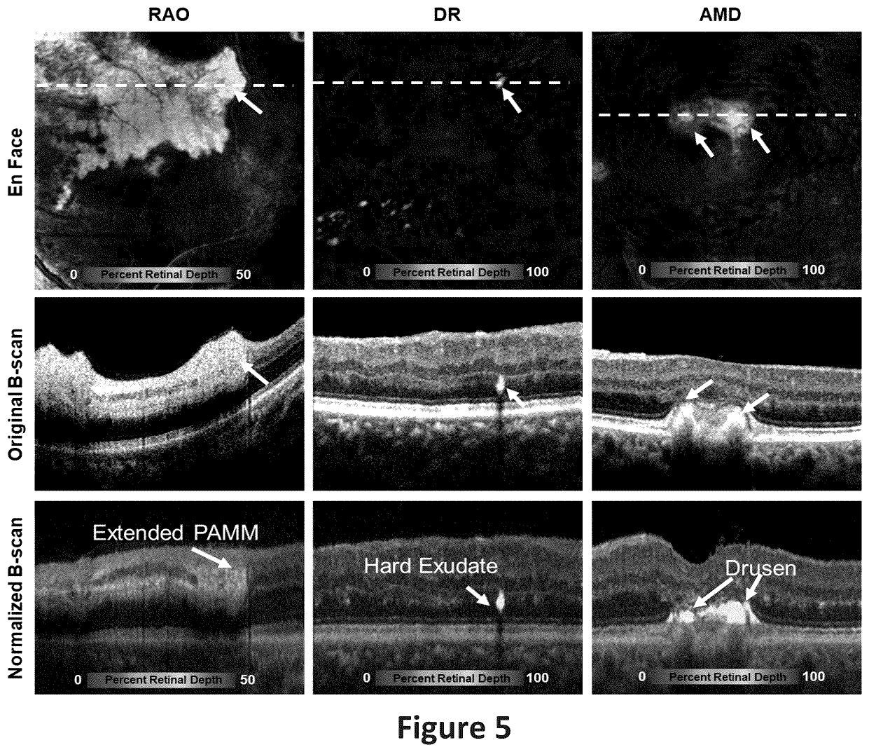

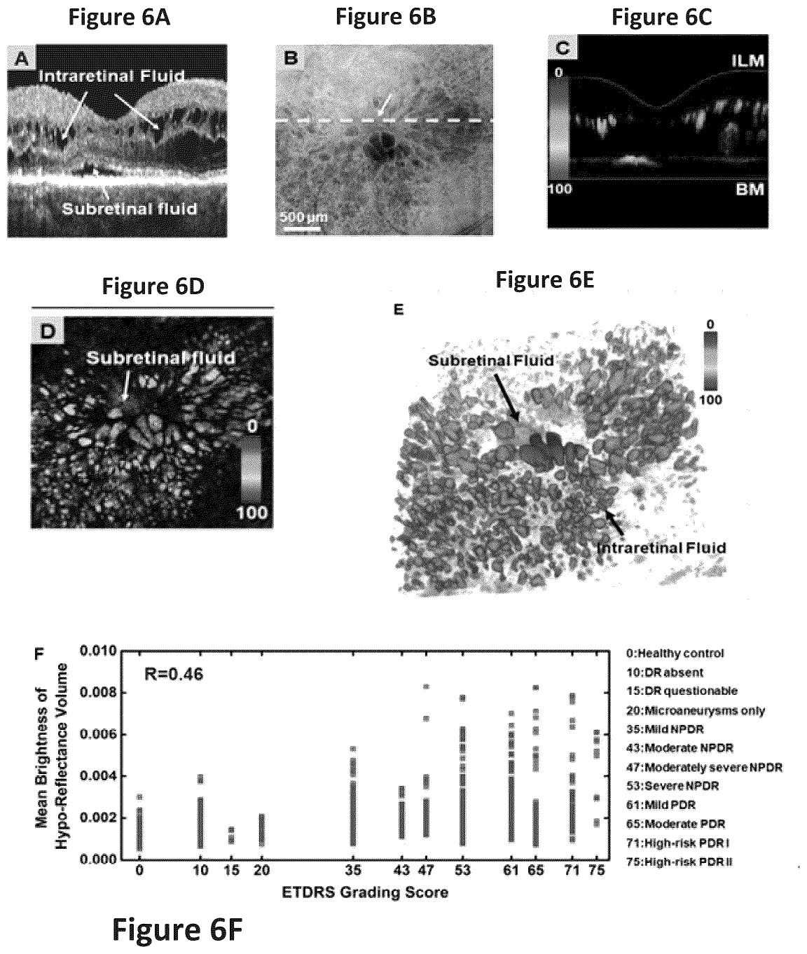

An interpretation of the standard retina as a probability map entails those regions in a target scan that deviate significantly from the standard are abnormal, which can be an indication of pathology. To enhance visualization of such pathologies, deviation image may be constructed in which the magnitude of a voxel gives the degree to which the target scan differs from the standard retina and its color is determined by percent depth. Using this approach, several important pathologies may be identified and clearly visualized. For example, paracentral acute middle maculopathy (PAMM) may be easily recognized with this approach. FIGS. 4A-4D illustrate detection of hyper-reflective PAMM in an eye with retinal artery occlusion (RAO), in accordance with an embodiment of the present disclosure. Compared to the structural en face image (FIG. 4B) generated from the single INL slab by an accurate layer boundary segmentation (which involves manual correction), the method according to the present disclosure delineated the exact same PAMM location with INL segmentation waived. The pathology detection in the deviation image was validated by measuring the degree to which PAMM co-occurred at the same location in typical structural OCT imaging (Jaccard similarity coefficient=0.85±0.08 in n=19 eyes with retinal artery occlusions; ground truth determined by thresholding with manual correction). Other pathologies, such as hard exudates in diabetic retinopathy (DR) and drusen in age-related macular degeneration (AMD) are also visually enhanced by the deviation image, as shown in FIG. 5. Furthermore, by color coding the percent retinal depth, the present approach can visually depth resolve pathologies. In one example of PAMM (e.g., RAO column of FIG. 5), the usefulness of depth information is demonstrated. Shown by color-coded depth, the extension of PAMM into the ganglion cell complex (GCC), which is otherwise difficult to see in the structural images, can be appreciated.

Beyond enhancing visualization of pathologic hypo-reflectivity, the hypo-reflectivity deviation volume can also be used to obtain useful quantifications. The mean magnitude of the deviation volume correlated with DR severity in 1069 scans (Spearman correlation to Early Treatment of Diabetic Retinopathy Study (ETDRS) score=0.46;

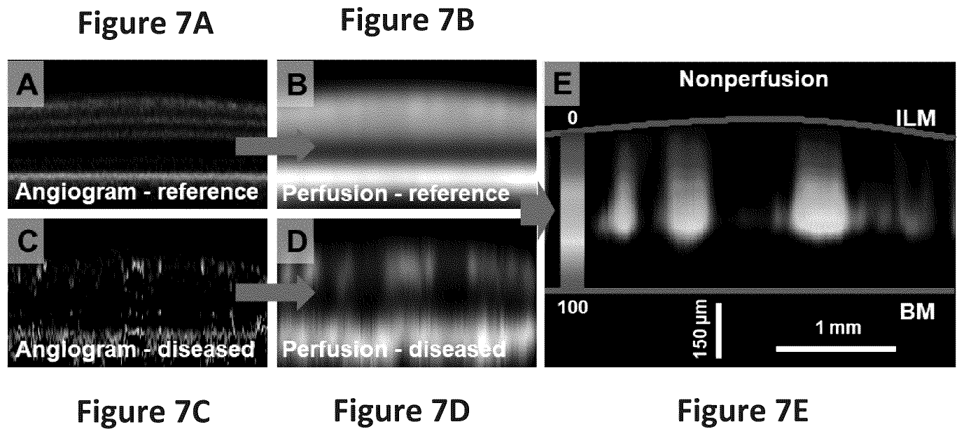

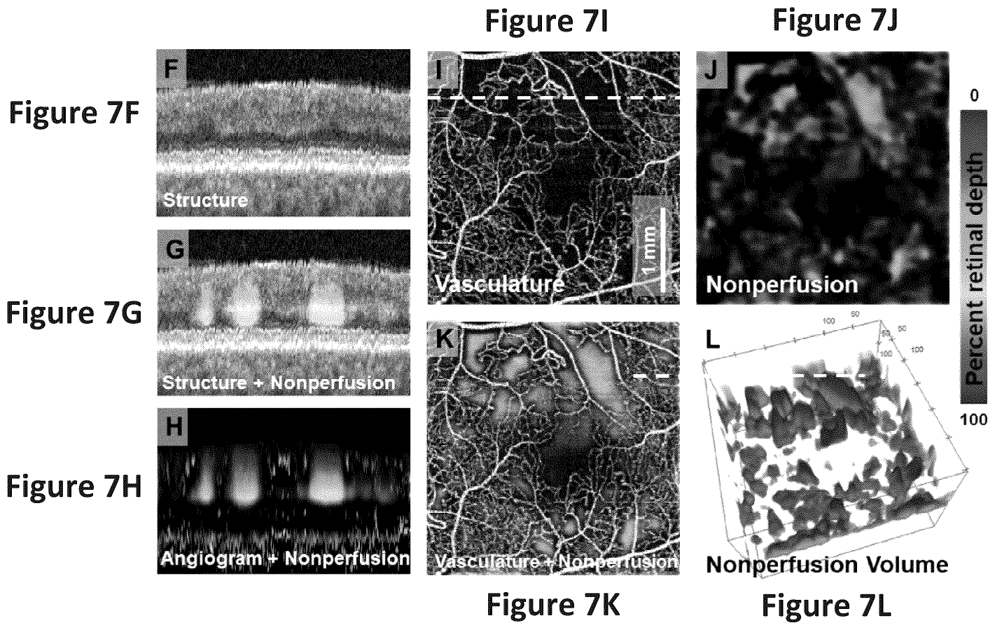

FIGS. 6A-6F), in one example. This metric-the average value of the hypo-reflectance deviation image, does not require segmentation or quantification of any pathologic features, and thus represents a conceptually simple and easy to use approach to quantify pathology.

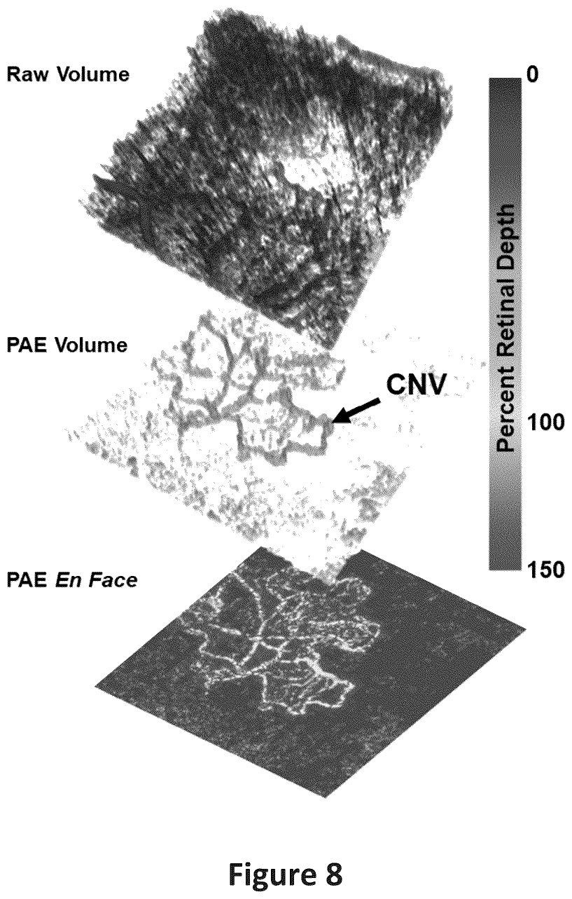

The ability of a vessel to perfuse a location depends on the distance between the two. The vessel perfusion may be modeled as a 3-D Gaussian kernel. Applying the Gaussian filter to the standard retina and target angiographic volume and constructing a deviation image highlights non-perfusion areas (e.g., as shown in FIGS. 7A-7D). The correspondence between the detected non-perfusion regions in the deviation image with no flow signal regions in standard OCTA may be verified by inspecting B-scans (FIGS. 7G-7H) and en face images (FIGS. 7I-7K). Similar to hypo-reflective pathology, the mean magnitude of the 3-D retinal non-perfusion deviation volume (FIG. 7L) achieved a Spearman correlation coefficient of 0.61 with the ETDRS score for the subjects in the same n=1069 OCT scans, in one example.

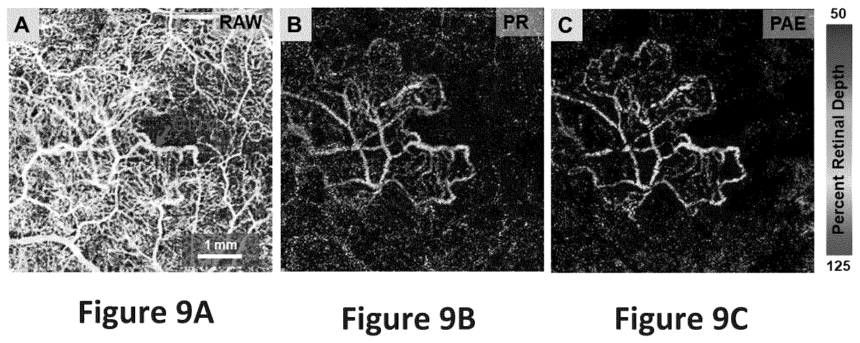

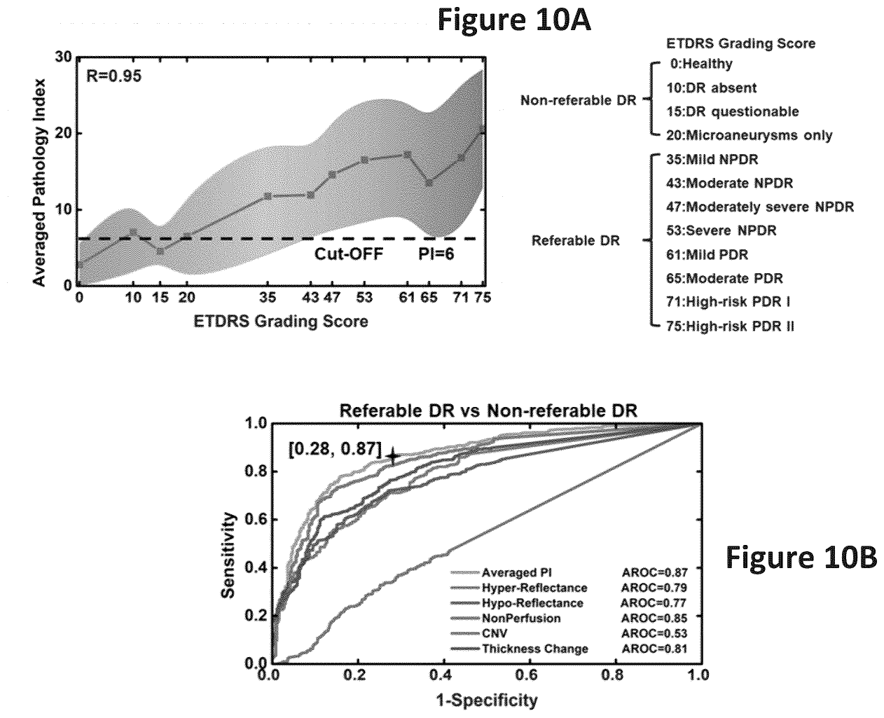

FIGS. 8A-8C depict visualization of choroidal neovascularization (CNV) in an eye with age-related macular degeneration (AMD) detected with a pathologic angiogenesis enhancement (PAE) algorithm. Since OCTA projection artifacts (e.g., in FIG. 9A) cast by anterior retinal circulation are removed by the projection-resolved OCTA (PR-OCTA) algorithm in this work, pathologic angiogenesis such as choroidal neovascularization (CNV) could be detected by comparing the target angiographic volume with the standard angiographic volume (FIG. 9B), with color indicating the CNV depth.

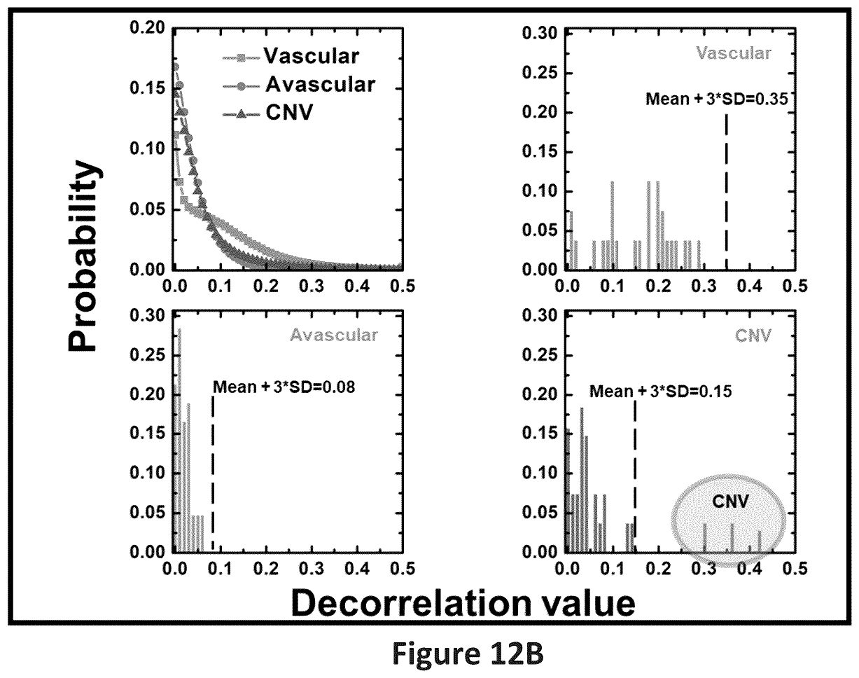

However, CNV is usually observed in the outer retina, which is avascular in healthy eyes. Due to its posterior location, CNV produces fewer projection artifacts than the normal retinal vessels in the raw (without projection artifacts removal) OCTA volume. Consequently, the distributions of the raw angiographic decorrelation value along the depth for each A-line can be categorized into three types: a half-normal distribution for avascular A-lines, a bipolar distribution for vascular A-lines, and a “disturbed half-normal” distribution for CNV A-lines (e.g., as shown in FIGS. 12A-12B). Here, by setting thresholds (Mean+3*SD) for the angiogram of each A-line, a pathologic angiogenesis enhancement (PAE) algorithm was developed to distinguish CNV voxels from raw OCTA volume (e.g., FIG. 8). The PAE angiographic volume could also be projected to a depth-coded en face image (e.g., FIG. 8 and FIG. 9C), which had comparable CNV visualization to an en face image made using PR-OCTA (FIG. 9B).

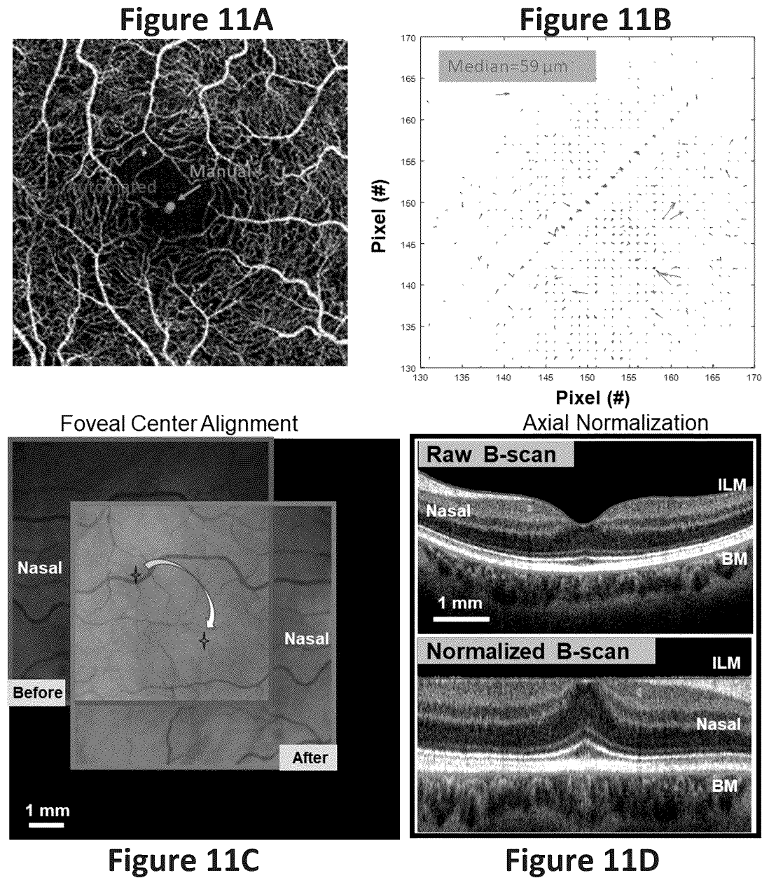

A pathology index (Eq. (1)) is constructed for each scan based on the magnitude of the voxel values in hypo-reflectance, hyper-reflectance, non-perfusion, CNV, and retinal thickness deviation volumes to the standard retina. Instead of focusing on quantification of a specific clinical feature, this framework is based on principles that could be applied indiscriminately to structural OCT and OCTA images of eyes with multiple diseases. The pathology index is calculated as the decibel ratio average across the five types of deviation features (hyper-reflectance, hypo-reflectance, non-perfusion, CNV, and retinal thickness change) in the target retina (Pa) to the average in healthy subjects (Ph; sampled from healthy eyes not used to produce the standard retina), followed with normalization between 0 to 40 dB. The effectiveness of the pathology index is verified using a dataset containing n=1069 scans of eyes with DR, in one example. The Spearman correlation coefficient for the pathology index with DR severity is 0.95, as shown in FIGS. 10A-10B. In addition, the area under the receiver operating characteristic curve (AROC) is 0.87 for the pathology index to differentiate referable DR from non-referable DR cases (FIGS. 10A-10B).

$$Pathology Index ( PI ) = 100 \times \frac{10 {log}_{10} {P}_{d} / {P}_{h} - 0}{40 - 0} \qquad ( 1 )$$

$${P}_{h} : averaged magnitude of pathology in healthy subjects \\ {P}_{d} : averaged magnitude of pathology in the diseased retina$$

Thus, OCT and OCTA provide micron resolution volumetric data sets. Currently, interpretation of OCT and OCTA scans rely on 2D images such as en face slab projections or cross-sections due to the lack of an effective 3D analytic framework. Several challenges impede the development of such a framework: (1) Population-based variation prevents direct comparison of data sets. Consequently OCT measurements like retinal thickness cannot be naively used to detect pathology, since unhealthy values may be normal in other individuals. (2) The reflectance signal that OCT relies on to generate tissue contrast can be influenced by opacities that affect the illumination pathway. This can cause variations in signal strength from scan to scan, and even region to region, which complicates analysis. (3) Current OCT/OCTA volumetric analyses are difficult to verify due to difficulties displaying volumetric data. This is especially problematic clinically, since professionals may need to verify algorithm outputs.

In the present disclosure, a novel framework is demonstrated for processing volumetric structural OCT and OCTA that can aid in disease agnostic detection and visualization of retinal pathologies. In embodiments, this framework relies on the construction of a standard retina by merging depth-normalized scans from healthy subjects. As shown in the results, a voxel-wise comparison between a target retina and the standard retina volumes for structure, angiography, and simulated 3D perfusion map can reveal pathology. Normalizing the depth between the ILM and BM helped reduce the effect of population variation in retinal thickness, which can confound disease detection. Normalization by depth also enables consistent color-mapping that can accurately locate pathology in 3 dimensions. In this way, intraretinal and subretinal fluid can be easily differentiated; these different fluid locations influence treatment response in diabetic macular edema. Additionally, a disease agnostic pathology index generated from this framework correlated with DR severity, indicating that this approach could also provide quantitative diagnostic value.

The described framework may provide several significant advantages. First, it may be volume-based and performed directly on volumetric scans. It has previously been reported that 3D vessel density demonstrates foveal ischemia more effectively than 2D methods (e.g., as described in Wang, B et al. Three-dimensional structural and angiographic evaluation of foveal ischemia in diabetic retinopathy: method and validation. Biomedical optics express 10, 3522-3532 (2019), incorporated by reference herein). It has also been reported that 3D retinal fluid can provide more comprehensive information than the 2D area (e.g., as described in Guo, Y et al. Automated segmentation of retinal fluid volumes from structural and angiographic optical coherence tomography using deep learning. Translational vision science & technology 9, 54-54 (2020), incorporated by reference herein). Second, the framework may be layer-independent and require minimal layer segmentation (e.g., just the ILM and BM borders are required). The ILM and BM are the easiest boundaries to segment. The techniques described herein may avoid the need to perform fine segmentations of retinal layers which can be a source of errors. Third, the techniques described herein may be disease agnostic and can cover a wide spectrum of clinical features.

Some embodiments may include additional features to further improve the results. First, with the ability to detect both morphological (hyper-reflectance, hypo-reflectance, retinal thickness change) and functional (non-perfusion and CNV) changes, the present method explores the possibility of establishing a pathology index. While the pathology index is described herein in the context of DR in the illustrated embodiment, this index may have clinical significance in a range of retinal diseases in other applications, since the features used to construct the index are not specific to DR. Second, as described herein, a standard retina may be constructed using data from several healthy individuals, representing a range of demographics. Sensitivity for pathology detection may be improved by using multiple standard volumes as disclosed herein that reflects demographic characteristics (such as age, sex, and ethnicity), instead of using a single generic standard. Finally, while the framework has been demonstrated in macular scans in the illustrated embodiments, the same approach may be adapted for other regions such as the optic disc in other embodiments.

Thus, the present disclosure establishes a 3D analytic framework to detect retinal pathologies with commercial OCT and OCTA by enabling volumetric comparisons of OCT scans from different subjects, with color coding by normalized depth in order to better appreciate the pathologies in projected en face images. The pathologies correlate well with DR severity and are visible in other diseases. The approach described herein provides a framework for studying structural OCT and OCTA in ophthalmology that can readily be integrated into commercial systems.

### EXAMPLES

The following examples are illustrative of the disclosed methods. In light of this disclosure, those skilled in the art will recognize that variations of these examples and other examples of the disclosed method would be possible without undue experimentation.

### Example 1

Purpose: To design a 3D analytic framework that can detect several retinal pathologies in three dimensions using structural and angiographic OCT requiring minimal retinal layer segmentation.

Human Subjects: Participants in this study were enrolled with written consent in accordance with an Institutional Review Board/Ethics Committee approved protocol at the Oregon Health & Science University and in accordance with the Declaration of Helsinki and compliant with the Health Insurance Portability and Accountability Act of 1996. As shown in Table 1, healthy participants (n=48, 6×6-mm scan), participants with diabetes (n=325, including 55 healthy controls), retinal artery occlusion (RAO, n=19), and age-related macular degeneration (AMD, n=142) were included in this study. All participants underwent a complete ophthalmic examination. For patients with diabetes, an Early Treatment of Diabetic Retinopathy Study (ETDRS) score was used to classify the severity into 12 subtypes and each eye underwent one or more imaging sessions at multiple follow up time points (ETDRS score may change at different follow up scans).

TABLE 1
Participant statistics

| | | Subject | Eye | Scan | |
|---|---|---|---|---|---|
| Participants | | (N) | (N) | (N) | SSI |
| Fellow Healthy Subjects | | 48 | 48 | 48 | 72 ± 7 |
| Diabetes | 0: Healthy control | 55 | 76 | 180 | 77 ± 8 |
| | 10: DR absent | 55 | 58 | 148 | 66 ± 8 |
| | 15: DR questionable | 2 | 2 | 5 | 72 ± 2 |
| | 20: Microaneurysms only | 10 | 11 | 26 | 69 ± 12 |
| | 35: Mild NPDR | 49 | 50 | 144 | 63 ± 9 |
| | 43: Moderate NPDR | 15 | 16 | 42 | 61 ± 8 |
| | 47: Moderately severe | 18 | 18 | 53 | 62 ± 7 |
| | NPDR | | | | |
| | 53: Severe NPDR | 53 | 57 | 150 | 61 ± 8 |
| | 61: Mild PDR | 49 | 52 | 155 | 63 ± 8 |
| | 65: Moderate PDR | 32 | 35 | 103 | 66 ± 8 |
| | 71: High-risk PDR I | 14 | 15 | 53 | 61 ± 9 |
| | 75: High-risk PDR II | 5 | 5 | 10 | 57 ± 4 |
| | Subtotal: | 325 | 374 | 1069 | 66 ± 10 |

| | | | | |
|---|---|---|---|---|
| Retinal Artery Occlusion | 19 | 19 | 19 | 57 ± 8 |
| (RAO) | | | | |
| Age-related Macular Degeneration | 142 | 280 | 431 | 57 ± 11 |
| (AMD) | | | | |

Abbreviations: diabetic retinopathy (DR), non-proliferative DR (NPDR), proliferative DR (PDR), signal strength index (SSI).

OCT and OCTA imaging: All optical coherence tomography (OCT) and OCT angiography (OCTA) scans were acquired by a commercial spectral domain OCT system (Avanti RTVue-XR, Optovue, Inc.). The light spectrum is centered at 840 nm with a full-width half-maximum bandwidth of 45 nm. The optical resolution in retina tissue is 5 μm and 15 μm in the axial and lateral directions, respectively. It covers a 3-mm imaging depth and provides a 3-μm digital sampling interval in the axial direction. The system operates in a 70-kHz A-line scanning rate with a 750 μW exposure power at the pupil. Inner limiting membrane (ILM) and Bruch's membrane (BM) boundaries were exported from the OCT system, which were calculated automatically using a directional graph search method (e.g., as described in Zhang, M. et al. Advanced image processing for optical coherence tomographic angiography of macular diseases. Biomedical Optics Express 6, 4661-4675 (2015), incorporated by reference herein). Compared to other boundaries, the ILM and BM are the easiest to segment, and generally do not require manual correction. Depth in this study was measured as the percent depth between these two boundaries (ILM=0; BM=100).

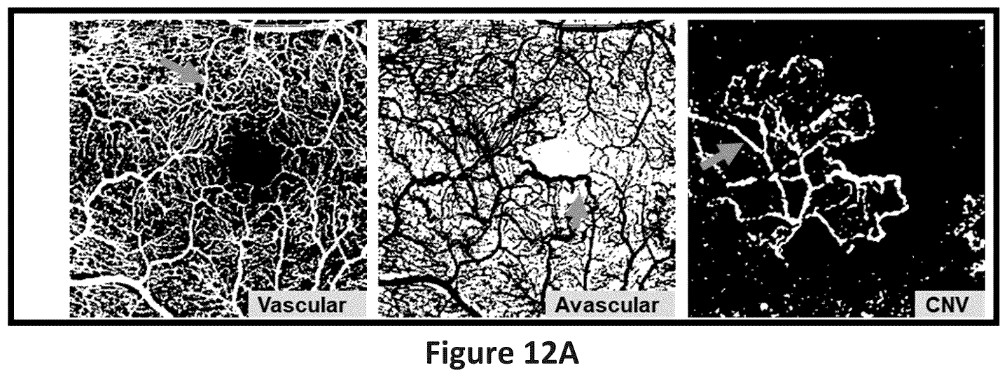

Scan registration in different subjects: This involved a two-step strategy, including the lateral alignment and depth normalization in the axial direction, to achieve the volumetric registration of scans in different subjects. As the foveal avascular zone (FAZ) is the most obvious feature in macular scans (e.g., as described in Chui, T Y et al. The association between the foveal avascular zone and retinal thickness. Investigative ophthalmology & visual science 55, 6870-6877 (2014), incorporated by reference herein), it was used to perform the lateral alignment, i.e. placing the FAZ in the exact center of a 9×9-mm image. The temporal raphe, part of the boundary between the superior and inferior hemispheres, was almost horizontal in all images, so no image rotations were needed to align the scans. Therefore, the lateral alignment was completed by detecting the center of the FAZ and then rigidly shifting the image to place the center of FAZ to the center of the image (FIGS. 11A-11D).

An automated method is described to locate the FAZ center without delineating the exact boundary of the FAZ. This involves 1) applying a gaussian filter (window size: 35×35) to the vascular en face images, 2) inverting the contrast, 3) multiplying by an image-centered gaussian weight map as the FAZ has a high probability of occurring in the center of the image, and 4) locating the FAZ center at the magnitude centroid of the image. As shown in FIG. 11B, the median distance from the automatically detected FAZ center to a manually labeled FAZ center was 59 μm for the scans used in this study. After that, foveal center alignment was performed by moving the FAZ centers to the center of a reference image in a zero-padded matrix, with the nasal and temporal sides flipped in left eyes to match the orientation in right eyes (FIG. 11C).

Next, axial normalization (ILM:0, BM:100) was performed to reduce inter-subject variation. This was achieved by 1) placing the ILM and BM boundaries at fixed depths (d₁, d₂), where d₁, d₂ can be constant over the entire retinal regions, or be dependent upon maps to retinal regions as long as the process is kept the same for all scans, 2) calculating the new depth for retinal tissue at z depth, and 3) interpolating the original A-lines according to the new depth to obtain the normalized A-lines (FIG. 11D, Eq. (2)). It should be noted that the process leads to heterogeneous axial resolution at different locations. In this study, to preserve the natural appearance of the retina on cross-sectional scans familiar to clinicians and researchers, the BM was placed at a constant depth d₂, while the ILM was placed at a region-dependent depth, which was determined by the mean retinal thickness maps in healthy subjects, i.e., the BM boundary was flat, and ILM boundary was curved (FIGS. 1, 2A-2B, 4A-4D, 6A-6F, and 7A-7L).

$$\{ \begin{matrix}{ILM}_{x , y} & \to & {d}_{1} \\ {Z}_{x , y} & \to & ( {Z}_{x , y} - {ILM}_{x , y} ) * ( {d}_{2} - {d}_{1} ) / ( {BM}_{x , y} - {ILM}_{x , y} ) + {d}_{1} \\ {BM}_{x , y} & \to & {d}_{2}\end{matrix} \qquad ( 2 )$$

Voxel-wise pathology detection: By registering and merging the scans in healthy subjects, standard retina volumes were obtained, including a structural, angiographic, and perfusion volumes. These standard retina volumes were further used as references for voxel-wise comparison to detect pathologies by registering the scans from diseased retinas according to the FAZ centering method (see above).

The variation of signal strength index (SSI) in the scans may be considered to compare the scans fairly, as SSI affected the OCT reflectance values as well as the angiogram values. Previously, it was difficult to eliminate the effect of SSI in the scans as image illumination was non-uniform. Here, by taking advantage of the standard retinas, the disclosed method effectively compensates the SSI volumetrically post-registration.

This was achieved by 1) 3-D filtering (down-sampling and followed by up-sampling with a factor of 10×) the reflectance of the scans and standardized retina, 2) calculating the 3-D reflectance ratio map for the filtered scan to the filtered standard retina, and 3) dividing the original scan by the ratio map. After that, the reflectance in the scan was brought to the same level as the standard retina in all regions. It should be noted that due to the shadow artifacts from vessels and hard exudates, the hypo reflectance was overestimated. To correct for this, it was necessary to further adjust the structural volume when measuring hypo-reflectance. This was achieved by 1) projecting the structural en face image from 20 μm above the BM to the BM for the sample volume and standardized volume, 2) calculating the reflectance ratio map of the two en face images, and 3) dividing the sample volume by the ratio map.

The pathology p was detected by the extent to which the scan from the diseased retina (x_{d}) deviated from the reference retina (xᵣ) through voxel-wise comparison, which was calculated from Eq. (3). Unless otherwise noted, the comparison method was the same for the quantification of thickness, reflectance, angiographic and perfusion volumes. The mean magnitude of pathology was calculated as the mean value of pathology volume p within the volumetric retinal tissue range. For better visualization, the pathologies were color-coded by both their amplitude and their percent retinal depth at specific voxels.

$$p = \frac{{( {x}_{d} - {x}_{r} )}^{2}}{{x}_{d}^{2} + {x}_{r}^{2}} \qquad ( 3 )$$

3-D Non-perfusion Map: It is known that neuronal retina tissue is supported by blood perfusion from the retinal and choroidal circulation. Several features are anticipated to influence the amount of perfusion from a vessel to a specific location: 1) perfusion is a three-dimensional process; 2) a capillary has maximal perfusion capability at its original location; and 3) perfusion capability decreases with increasing distance. Based on these features, the perfusion capability from a vascular voxel may be modeled as a 3-D Gaussian kernel c=exp(−r²/2/σ²), with c standing for perfusion capability, r for perfusion distance, and a for the standard deviation of the Gaussian kernel. The value of a was determined to be 40 μm based on the three-sigma rule by considering the ˜120-μm thickness of the avascular outer retina in humans (e.g., measured from the healthy subjects in this study).

It can be assumed that a voxel with larger flow value can provide stronger perfusion to retinal tissue, and more vascular voxels indicates more powerful perfusion. Based on these points, the 3-D blood perfusion map (P=A×C) was simulated by performing a convolution to the 3-D perfusion capability kernel C with the OCT angiography volume A. By comparing with reference values in heathy subjects as described previously, non-perfusion regions were detected in those voxels with values smaller than the reference, with the degree of non-perfusion quantified by Eq. (3) to evaluate how much it deviated from a healthy status.

Pathologic Angiogenesis Enhancement: In OCTA, projection artifacts in posterior layers can hinder visualization of CNV in the avascular outer retina (FIG. 8). A straightforward strategy for dealing with this complication is to remove the projection artifacts and clean up the outer retinal angiogram (e.g., as described in Zhang, M. et al. Projection-resolved optical coherence tomographic angiography. Biomedical optics express 7, 816-828 (2016), incorporated by reference herein). However, it is usually hard to differentiate the CNV flow signal from projection artifacts. In this study, we demonstrate a simpler approach to distinguish CNV voxels from the raw angiogram volume, without removing projection artifacts. The A-lines in a scan volume can be categorized into three types: normal vascular A-lines, avascular A-lines, and the A-lines with CNV (FIG. 12A). For the vascular A-lines, decorrelation values along the depth are largest in the vessel voxels, but also present in layers with strong reflectance. For those layers with weak reflectance, the decorrelation values are minimal and close to 0. This feature causes a bipolar distribution for the decorrelation values in normal vascular A-lines (FIG. 12B—Vascular). For the avascular A-lines, the decorrelation values are minimal across the entire depth, forming a half-normal distribution centered at 0 (FIG. 12B—Avascular). However, for the CNV A-lines, the voxels above the CNV are similar to the avascular voxels with the half-normal distribution, and only a few voxels at and posterior to CNV locations have significantly larger decorrelation values. This is called a “disturbed half-normal distribution” (FIG. 12B—CNV). The features can be better appreciated in the representative A-lines (FIG. 12B).

By taking advantage of the distribution of features, a simple approach to identify the CNV voxels is to threshold each A-line using angiogram cutoffs (mean+3*SD) calculated from individual A-lines. For the vascular A-lines, the threshold is very large (0.35 for the representative A-line in FIG. 12B). For the avascular A-lines, the threshold is small (0.08 for the representative A-line in FIG. 12B). The decorrelation values of all voxels in those two types of A-lines are below the mean+3*SD threshold. However, the CNV voxels behave as outliers, superior to the thresholds, and thus can be identified in the raw angiogram volume.

Conclusions: The 3D analytic framework can be used to detect and visualize several retinal pathologies in three dimensions with minimal retinal layer segmentation. This framework could be integrated into commercial OCT systems to potentially improve ophthalmic research and clinical care.

### Example 2

The methods described in the present disclosure may be implemented in an integrated system that is fully automated or assembled from different components that may require some manual intervention. In general, a system according to the present disclosure may comprise the components of a corneal topography measuring device capable of measuring and generating a corneal topography and an optical coherence tomography device, wherein both devices are capable of producing data in digital format or in a format that can be digitized, and a processing unit. The corneal topography measuring device may include, but not be limited to, Placido-ring topography, slit-scan corneal topography, Shiempflug-camera corneal tomography, raster photogrammetry, optical coherence tomography, or any other suitable cornea measuring devices known in the art. The processing unit may be a personal computer, a workstation, an embedded processor, or any other suitable data processing device commonly known in the art.

In addition to being implemented in a system, the methods of the present disclosure may also be provided in the form of software encoded on a computer readable medium for distribution to end users. Example computer media may include, but not be limited to, floppy disks, CD-roms, DVDs, hard drive disks, flash memory cards, downloadable files on an internet accessible server, or any other computer readable media commonly known in the art.

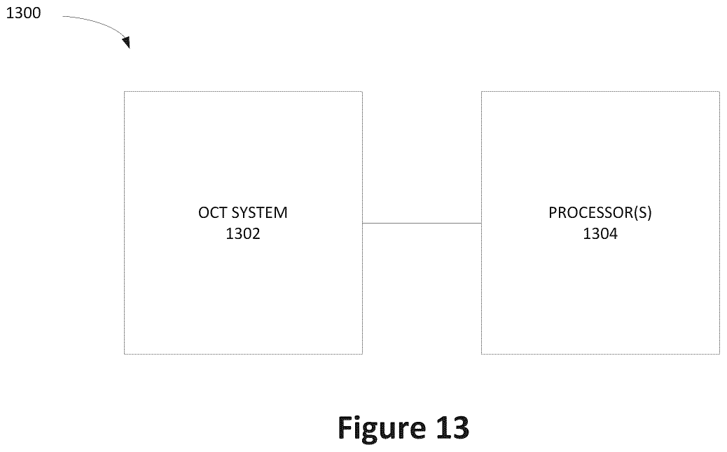

FIG. 13 schematically shows an example system 1300 for OCT image processing in accordance with various embodiments. System 1300 comprises an OCT system 1302 configured to acquire an OCT image comprising OCT interferograms and one or more processors or computing systems 1304 that are configured to implement the various processing routines described herein. OCT system 1300 can comprise an OCT system suitable for OCT angiography applications, e.g., a swept source OCT system or spectral domain OCT system.

In various embodiments, an OCT system can be adapted to allow an operator to perform various tasks. For example, an OCT system can be adapted to allow an operator to configure and/or launch various ones of the herein described methods. In some embodiments, an OCT system can be adapted to generate, or cause to be generated, reports of various information including, for example, reports of the results of scans run on a sample.

In embodiments of OCT systems comprising a display device, data and/or other information can be displayed for an operator. In embodiments, a display device can be adapted to receive an input (e.g., by a touch screen, actuation of an icon, manipulation of an input device such as a joystick or knob, etc.) and the input can, in some cases, be communicated (actively and/or passively) to one or more processors. In various embodiments, data and/or information can be displayed, and an operator can input information in response thereto.

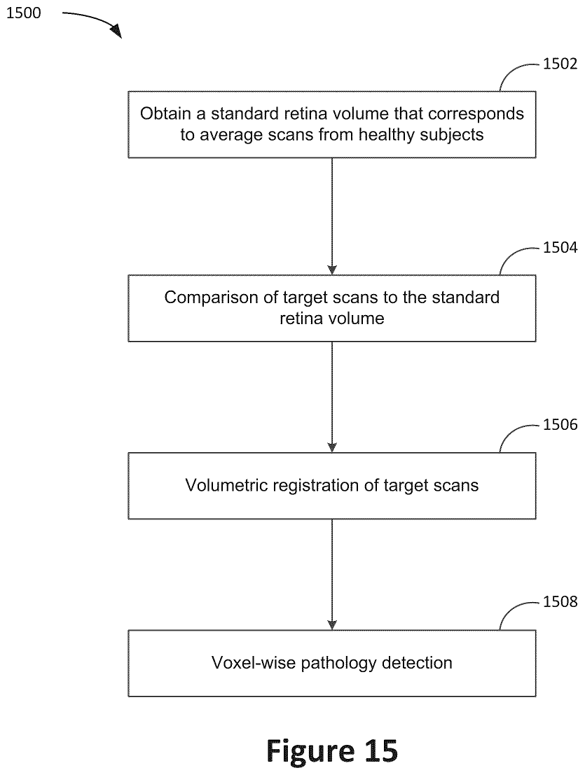

In some embodiments, the above described methods and processes can be tied to a computing system, including one or more computers. In particular, the methods and processes described herein, e.g., the method depicted in FIG. 15 described below, can be implemented as a computer application, computer service, computer API, computer library, and/or other computer program product.

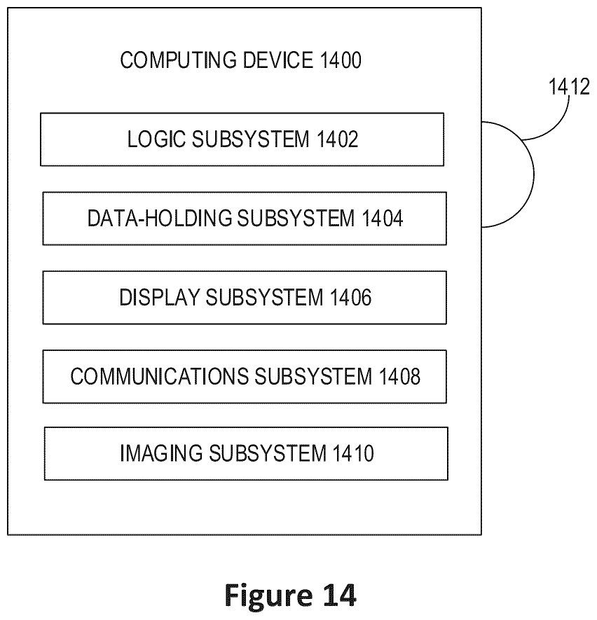

FIG. 14 schematically shows a non-limiting computing device 1400 that can perform one or more of the methods and processes described herein. For example, computing device 1400 can represent the processor 1304 included in system 1300 described above, and can be operatively coupled to, in communication with, or included in an OCT system or OCT image acquisition apparatus. Computing device 1400 is shown in simplified form. It is to be understood that virtually any computer architecture can be used without departing from the scope of this disclosure. In different embodiments, computing device 1400 can take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1400 includes a logic subsystem 1402 and a data-holding subsystem 1404. Computing device 1400 can optionally include a display subsystem 1406, a communication subsystem 1408, an imaging subsystem 1410, and/or other components not shown in FIG. 14. Computing device 1400 can also optionally include user input devices such as manually actuated buttons, switches, keyboards, mice, game controllers, cameras, microphones, and/or touch screens, for example.

Logic subsystem 1402 can include one or more physical devices configured to execute one or more machine-readable instructions. For example, the logic subsystem can be configured to execute one or more instructions that are part of one or more applications, services, programs, routines, libraries, objects, components, data structures, or other logical constructs. Such instructions can be implemented to perform a task, implement a data type, transform the state of one or more devices, or otherwise arrive at a desired result.

The logic subsystem can include one or more processors that are configured to execute software instructions. For example, the one or more processors can comprise physical circuitry programmed to perform various acts described herein. Additionally or alternatively, the logic subsystem can include one or more hardware or firmware logic machines configured to execute hardware or firmware instructions. Processors of the logic subsystem can be single core or multicore, and the programs executed thereon can be configured for parallel or distributed processing. The logic subsystem can optionally include individual components that are distributed throughout two or more devices, which can be remotely located and/or configured for coordinated processing. One or more aspects of the logic subsystem can be virtualized and executed by remotely accessible networked computing devices configured in a cloud computing configuration.

Data-holding subsystem 1404 can include one or more physical, non-transitory, devices configured to hold data and/or instructions executable by the logic subsystem to implement the herein described methods and processes. When such methods and processes are implemented, the state of data-holding subsystem 1404 can be transformed (e.g., to hold different data).

Data-holding subsystem 1404 can include removable media and/or built-in devices. Data-holding subsystem 1404 can include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1404 can include devices with one or more of the following characteristics: volatile, nonvolatile, dynamic, static, read/write, read-only, random access, sequential access, location addressable, file addressable, and content addressable. In some embodiments, logic subsystem 1402 and data-holding subsystem 1404 can be integrated into one or more common devices, such as an application specific integrated circuit or a system on a chip.

FIG. 14 also shows an aspect of the data-holding subsystem in the form of removable computer-readable storage media 1412, which can be used to store and/or transfer data and/or instructions executable to implement the herein described methods and processes. Removable computer-readable storage media 1412 can take the form of CDs, DVDs, HD-DVDs, Blu-Ray Discs, EEPROMs, flash memory cards, USB storage devices, and/or floppy disks, among others.

When included, display subsystem 1406 can be used to present a visual representation of data held by data-holding subsystem 1404. As the herein described methods and processes change the data held by the data-holding subsystem, and thus transform the state of the data-holding subsystem, the state of display subsystem 1406 can likewise be transformed to visually represent changes in the underlying data. Display subsystem 1406 can include one or more display devices utilizing virtually any type of technology. Such display devices can be combined with logic subsystem 1402 and/or data-holding subsystem 1404 in a shared enclosure, or such display devices can be peripheral display devices.

When included, communication subsystem 1408 can be configured to communicatively couple computing device 1400 with one or more other computing devices. Communication subsystem 1408 can include wired and/or wireless communication devices compatible with one or more different communication protocols. As non-limiting examples, the communication subsystem can be configured for communication via a wireless telephone network, a wireless local area network, a wired local area network, a wireless wide area network, a wired wide area network, etc. In some embodiments, the communication subsystem can allow computing device 1400 to send and/or receive messages to and/or from other devices via a network such as the Internet.

When included, imaging subsystem 1410 can be used to acquire and/or process any suitable image data from various sensors or imaging devices in communication with computing device 1400. For example, imaging subsystem 1410 can be configured to acquire OCT image data, e.g., interferograms, as part of an OCT system, e.g., OCT system 1302 described above. Imaging subsystem 1410 can be combined with logic subsystem 1402 and/or data-holding subsystem 1404 in a shared enclosure, or such imaging subsystems can comprise periphery imaging devices. Data received from the imaging subsystem 1410 can be held by data-holding subsystem 1404 and/or removable computer-readable storage media 1412, for example.

FIG. 15 provides the operations of an example method 1500 for detecting retinal pathologies, in accordance with various embodiments of the present disclosure. Method 1500 can be implemented by a system such as system 1300 described above, that includes an OCT system and one or more processors or computing systems, such as computing device 1400 described above. For example, one or more operations described herein can be implemented by one or more processors having physical circuitry programmed to perform the operations. In embodiments, one or more steps of method 1500 can be automatically performed by one or more processors or computing devices. Further, various acts illustrated in FIG. 15 can be performed in the sequence illustrated, in other sequences, in parallel, or in some cases omitted. Method 1500 can be used to enact a 3-D analytic framework for processing volumetric structural OCT and OCTA that can aid in disease agnostic detection and visualization of retinal pathologies.

In operation 1502, method 1500 includes to obtain a standard retina volume that corresponds to average scans from healthy subjects. In some embodiments, the standard retina volume may be constructed by merging and averaging depth-normalized OCT scans from healthy subjects. For example, OCT scan data including a plurality of interferograms may be acquired from a swept-source or other OCT system, e.g., the system shown in FIG. 1300. In other embodiments, the OCT data may be received by a computing device from an OCT scanning system via a network or from a storage medium coupled to or in communication with the computing device. For example, the standard retina volume may include a structural, angiographic, and/or perfusion volume.

In some embodiments, a predetermined standard retina volume may be stored and/or otherwise accessible by the system. For example, the standard retina volume may have been generated by another OCT system (or multiple OCT systems) and provided to the system that performs the method 1500. In other embodiments, the local system that performs the method 1500 may generate the standard retina volume and store the standard retina volume for use in the method 1500.

In operation 1504, method 1500 includes comparison of target scans to the standard retina volume constructed in operation 1502. The target OCT scan data, in embodiments, may be acquired from a swept-source or other OCT system, e.g., the system shown in FIG. 1300 as described above. In examples, the target scans may include OCT scans from diseased retinas for comparison with the standard retina volume.

In operation 1506, method 1500 includes volumetric registration of target scans. In embodiments, the volumetric registration of scans in different subjects may be achieved by a two-step strategy. The two-step strategy may include lateral alignment of the foveal center and depth normalization of A-lines in an axial direction in the target scans, for example. In some examples, the scans from diseased retinas may be registered according to the FAZ centering method.

In operation 1508, method 1500 includes voxel-wise pathology detection. In embodiments, a voxel-wise comparison between a target retina and the standard retina volumes for structure, angiography, and simulated 3D perfusion map can reveal pathology based on detected deviations. In some examples, a deviation image may be constructed in accordance with the present disclosure in which the magnitude of a voxel gives the degree to which the target scan differs from the standard retina. Additionally, a pathology index measuring the average deviation of a target retina from the standard may provide quantitative analysis.

It is to be understood that the configurations and/or approaches described herein are exemplary in nature, and that these specific embodiments or examples are not to be considered in a limiting sense, because numerous variations are possible. The specific routines or methods described herein can represent one or more of any number of processing strategies. As such, various acts illustrated can be performed in the sequence illustrated, in other sequences, in parallel, or in some cases omitted. Likewise, the order of the above-described processes can be changed.

The subject matter of the present disclosure includes all novel and nonobvious combinations and sub-combinations of the various processes, systems and configurations, and other features, functions, acts, and/or properties disclosed herein, as well as any and all equivalents thereof.

What is claimed is:

1. A computer-based method for detecting retinal pathology, the method comprising:
comparing voxel-wise a target scan of a retina to a reference retina volume to detect one or more deviations; and
detecting one or more retinal pathologies in the retina based on the one or more deviations.

2. The method of claim 1, wherein the reference retina volume is constructed by merging and averaging a plurality of depth-normalized scans from healthy subjects.

3. The method of claim 2, wherein the target scan is acquired using optical coherence tomography (OCT).

4. The method of claim 1, wherein the voxel-wise comparison of the target scan with the reference retina volume includes comparing structural information, angiography information, and/or a simulated three-dimensional perfusion map.

5. The method of claim 1, further comprising constructing a deviation image based on the detected one or more deviations, wherein a magnitude of respective voxels of the deviation image indicates a degree to which the target scan deviates from the reference retina volume.

6. The method of claim 1, further comprising determining a pathology index which indicates an average deviation of the target scan from the reference retina volume.

7. The method of claim 6, wherein the average deviation includes an average of two or more of hypo-reflectance, hyper-reflectance, non-perfusion, choroidal neovascularization, and retinal thickness deviation volumes.

8. A computer-based method for detecting retinal pathology, the method comprising:
obtaining a target scan of a retina via an optical coherence tomography (OCT) system;
registering the target scan volumetrically;
comparing voxel-wise the target scan to a standard retina volume to detect one or more deviations, wherein the standard retina volume corresponds to an average of a plurality of depth-normalized scans from healthy subjects; and
detecting one or more retinal pathologies in the based retina on the detected one or more deviations.

9. The method of claim 8, wherein the registration of the target scan includes lateral alignment of a foveal center and depth normalization of A-lines in an axial direction in the target scan.

10. The method of claim 9, further comprising segmenting an inner limiting membrane (ILM) boundary and a Bruch's membrane (BM) boundary of the target scan.

11. The method of claim 8, wherein the voxel-wise comparison of the target scan with the standard retina volume includes comparing structural data, angiography data, and/or a simulated three-dimensional perfusion map.

12. The method of claim 8, further comprising determining a pathology index which is calculated as a decibel ratio average across deviation in two or more of hypo-reflectance, hyper-reflectance, non-perfusion, choroidal neovascularization, and retinal thickness in the target scan to an average in healthy subjects.

13. The method of claim 8, wherein the detecting one or more retinal pathologies includes detecting abnormal reflectivity, neovascularization, and/or non-perfusion.

14. One or more non-transitory, computer-readable media (NTCRM) having instructions, stored thereon, that when executed by one or more processors of an optical coherence tomography (OCT) system cause the OCT system to:
obtain a target scan of a retina;
compare voxel-wise the target scan to a reference retina volume to detect one or more deviations; and
detect one or more retinal pathologies in the retina based on the one or more deviations.

15. The one or more NTCRM of claim 14, wherein the reference retina volume corresponds to an average of a plurality of depth-normalized scans from healthy subjects.

16. The one or more NTCRM of claim 14, wherein the voxel-wise comparison of the target scan with the standard retina volume includes to compare structural information, angiography information, and/or a simulated three-dimensional perfusion map.

17. The one or more NTCRM of claim 14, wherein the instructions, when executed, further cause the OCT system to generate a deviation image based on the detected one or more deviations, wherein a magnitude of respective voxels of the deviation image indicates a degree to which the target scan deviates from the reference retina volume.

18. The one or more NTCRM of claim 14, wherein the instructions, when executed, further cause the OCT system to determine a pathology index which indicates an average deviation of the target scan from the reference retina volume.

19. The one or more NTCRM of claim 18, wherein the average deviation includes an average deviation of two or more of hypo-reflectance, hyper-reflectance, non-perfusion, choroidal neovascularization, and retinal thickness.

20. The one or more NTCRM of claim 14, wherein to detect one or more retinal pathologies includes to detect abnormal reflectivity, neovascularization, and/or non-perfusion.
